(19) United States
(12) Patent Application Publication
(10) Pub. No.: US 2011/0106111 A1
(43) Pub. Date: May 5, 2011

(54) Suture Fixation Arthroscope Apparatus of the Temporomandibular Joint Disk

(75) Inventors: Chi Yang, Shanghai City (CN); Shanyong Zhang, Shanghai City (CN); Zhiyuan Zhang, Shanghai City (CN); Minjie Chen, Shanghai City (CN); Xieyi Cai, Shanghai City (CN); Jinning Zhang, Shanghai City (CN); Xiuming Liu, Shanghai City (CN)

(73) Assignee: Shanghai Ninth People's Hospital Affiliated to Shanghai Jiao Tong University School of Medicine, Shanghai City (CN)

(21) Appl. No.: 12/992,085

(22) PCT Filed: Feb. 5, 2010
(86) PCT No.: PCT/CN2010/070546

§ 371 Date: Nov. 10, 2010

(30) Foreign Application Priority Data
Apr. 14, 2009 (CN) 200910049274.9

(51) Int. Cl. A61B 17/04

(52) U.S. Cl. 606/147

(57) ABSTRACT

A suture fixation arthroscope apparatus of the temporomandibular joint (TMJ) disk comprises a first suture needle and a first inner core, a second suture needle and a second inner core, and a third suture needle. The tail ends of the first and second suture needles are connected with a big handle via a valve panel, a vertical groove is opened towards the center of a circle of the surface of the big handle, a horizontal groove which is the same size as the vertical groove is vertically set at the place of the big handle connecting the valve panel. The first inner core includes a seamless tube and a top welded with wire snares, the second inner core includes a seamless tube and a top welded with a crochet hook, the radian near the tails of the first and second inner cores is 90 degrees, a small handle is connected at the tails. The first and second inner cores can pass through the vertical groove of the big handle of the first and second suture needles respectively, the corner of the 90 degrees tail is able to lock rightwards or leftwards at the horizontal groove, the wire snare or the crochet hook at the top of inner cores are able to come out from the top of the suture needles.

### FIELD OF THE INVENTION

The present invention relates to a field of medical treatment apparatus, and more particularly to say, refers to an suture fixation arthroscope apparatus of the disk of temporomandibular joint (TMJ).

### BACKGROUND OF THE INVENTION

Since 1990s, there has been some scholars trying to use the minimally invasive surgery (inner mirror) to cure the shift of the disk of temporomandibular joint (TMJ). The common ground of all the methods is using the traction suture to the back zone of the joint disk, in order to recover the joint disk to the normal position, however, the imaging text displayed after the surgery: most of the joint disks had not been restored. Since 1990s, the present inventor followed the lead of the representative methods of U.S. (McCain, 1992) and Japan (Ohnishi, 1991) one after the other, but more than 90% of TMJ disk could not be recovered to the normal position. These accelerated us to reconsider the scientificity of the surgery itself, to find the drawbacks of the design, mostly represented as: (1) it was not completely the same between the direction of the suture traction and the long axis of the forward-backward orientation of the shifted joint disk; and (2) it was all used mongline suture, and the mongline suture was positioned at ⅓ of the joint disk outwards, therefore, it was unable to stabilize the joint disk whose inner and outer diameter are 2 cm to the normal position. In order to conquer the drawbacks, the present inventor prepared a suture apparatus which is applicable to the joint disk restoration fixing surgery under the new joint mirror.

In the restoration fixing surgery of the TMJ disk, due to lack of appropriate surgery apparatus, this kind of surgery has no good progress, currently the calculi forceps is adopted to pull out the seam in clinic, the method is less likely to completely lock up the seam with the suture needle, the seam is more likely to slip during a surgery, leading the same actions are repeated in the surgery, adding pain to the patients, the suture needle has no handles which is hard to operate.

### SUMMARY OF THE INVENTION

An object of the present invention is to provide a suture restoration fixing apparatus of the disk of TMJ which is adapted to the joint disk restoration fixing surgery under the new joint mirror, easy to operate, less likely to slip during a surgery.

Therefore, the technical solution of the present invention is disclosed as followed. A suture fixation arthroscope apparatus of the temporomandibular joint (TMJ) disk, characterized in that: comprising a first suture needle and a first inner core, a second suture needle and a second inner core and a third suture needle, wherein:

The tail of the first suture needle is connected with a big handle via a valve panel, a vertical groove is opened towards a circle center of the surface of the big handle, a horizontal groove which is the same size as the vertical groove is vertically disposed in the place of the big handle connecting the valve panel, the first inner core comprises a seamless tube having an inner diameter less than the first suture needle, a wire snare protruding at both ends is welded at the top of the seamless tube, the radian angle is 90 degrees near the tail of seamless tube, a small handle is connected at the tail of the seamless tube, the first inner core can pass through the vertical groove of the big handle of the first suture needle, the corner of the 90 degrees near the tail of the seamless tube is able to lock rightwards or leftwards at the horizontal groove, and the wire snare at the top of the seamless tube exposes from the top of the first suture needle; the tail of the second suture needle is connected with another big handle via a valve panel, a vertical groove is opened towards the circle center of the surface of the big handle, a horizontal groove which is the same size as the vertical groove is vertically disposed in at the place of the big handle connecting the valve panel, the second inner core comprises a seamless tube having inner diameter less than the second suture needle, a crochet hook the same size as the inner diameter is welded at the top of the seamless tube, the radian angle is 90 degrees near the tail of the seamless tube, a small handle is connected at the tail of the seamless tube, the second inner core can pass through the vertical groove of the big handle of the second suture needle, the corner of the 90 degrees near the tail of the seamless tube is able to lock rightwards or leftwards at the horizontal groove, and the crochet hook at the top of the seamless tube exposes from the top of the second suture needle; and the third suture needle is a normal No. 12 puncture needle.

The inner diameter of the first protruded end of the two protruded ends wire snare is smaller than the body of the seamless tube, and the inner diameter of the second protruded end is greater than the body of the seamless tube; the inside of the hook of the crochet hook can exactly fit one seam.

The basis material of the seam is made of polyester fiber knitting thread 2-0, at least comprises a guiding zone and a functional zone, the guiding zone located at one or two sides of the seam is processed by the adhesive, and the functional zone is not processed by the adhesive, keeping the original pliability. The guiding zone and the functional zone can distinguish by colors.

The present invention can also include a small hole is in the middle of the small handle at the tail of the inner core.

The present invention has the profitable efforts thereafter.

The big handles of the first and second suture needles are convenient to operate in a surgery, the horizontal groove on it which is perpendicular to the circular channel can lock the inner core apropos, and the first inner core of the springe-at-top steel wire snare replaces the former calculi forceps, and is more likely to completely lock up the seam with the suture needle, the seam is less likely to slip during the surgery, the crochet hook at the top of the second inner core can contain the seam apropos, thereby lock the seam between the crochet hook and the suture needle, the seam can be pull out during the surgery, and be not easy to loose, the present invention makes the surgery processed successfully by one time, rather than repeating one surgery action, greatly releasing the pain of the patient.

### DESCRIPTION OF THE DRAWINGS

Accompanying with the drawings of the description, a further detailed description to the invention is described below.

FIG. 1A is the first suture needle, FIG. 1B is the first inner core, and FIG. 1C is the state of the first suture needle and the first inner core combining using according to one embodiment of the present invention;

FIG. 2A is the second suture needle, FIG. 2B is the second inner core, and FIG. 2C is the state of the second suture needle and the second inner core combining using according to one embodiment of the present invention;

FIG. 3 is a schematic view of the third suture needle according to one embodiment of the present invention;

FIG. 4 is a schematic enlarged view of the springe-at-top steel wire snare of the first inner core according to one embodiment of the present invention; and

FIG. 5 is a schematic enlarged view of the crochet hook at the top of the second inner core according to one embodiment of the present invention.

### DETAILED DESCRIPTION OF THE PREFERRED EMBODIMENTS

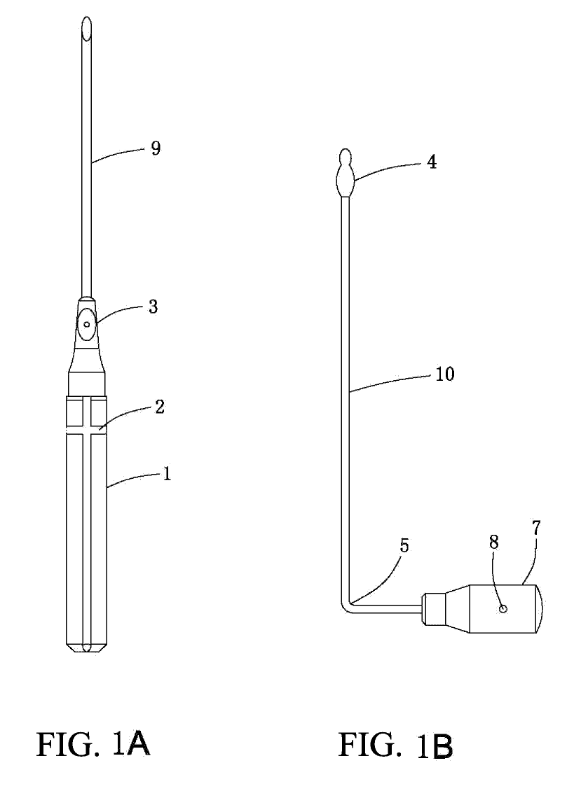

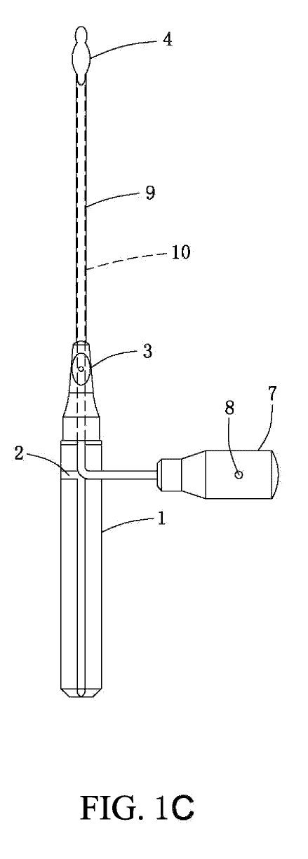

FIG. 1A is the first suture needle, made from No. 12 puncture needle, at the tail of suture needle 9 via the valve panel 3 connected the columned big handle 1, a vertical groove was opened towards the center of a circle of the surface of the big handle 1, a horizontal groove 2 which had the same size with the vertical groove was vertically set at the place of the big handle 1 near the valve panel 3; FIG. 1B is the first inner core, including a seamless tube 10 whose inner diameter is less than the first suture needle 9, a two-end-protuberant wire snare 4 is jointing to the top of seamless tube 10, the angle 5 of the radian is 90 degrees near the tail of seamless tube 10, a small handle 7 is connected at the tail; FIG. 1C is the state of the first suture needle and the first inner core combining using, the first inner core could pass through the vertical groove of the big handle 1 of the first suture needle, the angle 5 of the 90 degrees tail is able to lock rightwards or leftwards at the horizontal groove 2, the wire snare 4 at the top is able to come out from the top of the first suture needle 9.

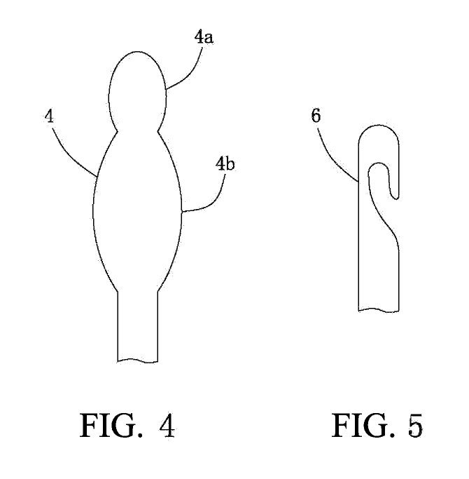

FIG. 4 is the zoom-in picture of the wire snare 4, the inner diameter of the first-end protuberance 4a is smaller than the body of the seamless tube, and the inner diameter of the second-end protuberance 4b is greater than the body of the seamless tube, the wire snare 4 unitarily possessed elasticity, the smaller inner diameter of the first-end protuberance 4a is convenient for itself to pass through the circular channel from the bottom of the big handle 1, the greater inner diameter of the second-end protuberance 4b is convenient for passing through the seam during a surgery.

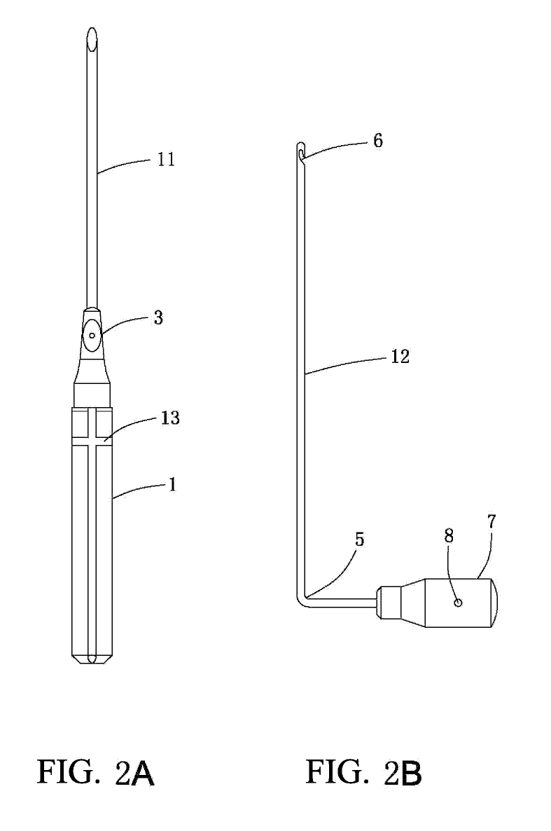

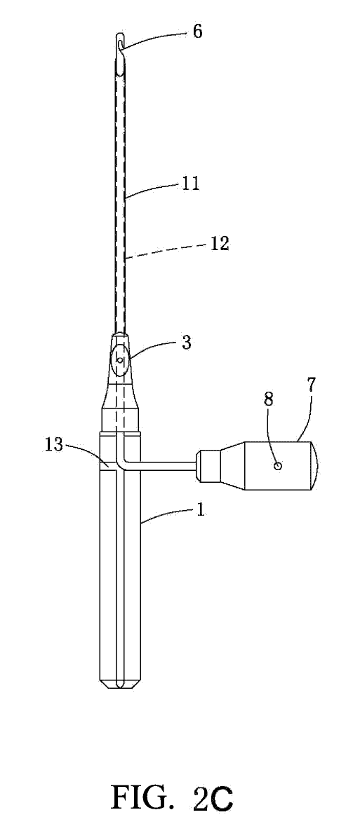

FIG. 2A is the second suture needle, and is also made from No. 12 puncture needle, likewise, at the tail of suture needle 11 via the valve panel 3 connected the columned big handle 1, a vertical groove is opened towards the center of a circle of the surface of the big handle 1, a horizontal groove 13 which had the same size with the vertical groove is vertically set at the place of the big handle 1 near the valve panel 3, the different point against the first suture needle is, due to the inner diameter at the top of the inner core (FIG. 1B, FIG. 2B) which is integrated used with it is inconsistent, correspondingly the sizes of the suture needle, the vertical groove, the horizontal groove were different (the inner diameter of the first suture needle 9 is slightly greater than the inner diameter of the second suture needle 11), the rest were the same; FIG. 2B is the second inner core, including a seamless tube 12 whose inner diameter is less than the second suture needle 11, a crochet hook having inner diameter is the same is welded at the top of seamless tube 12, the angle 5 of the radian is 90 degrees near the tail of seamless tube 12, a small handle 7 is connected at the tail; FIG. 2C is the state of the second suture needle and the second inner core combining using, the second inner core could pass through the vertical groove 13 of the big handle 1 of the second suture needle, the angle 5 of the 90 degrees tail is able to lock rightwards or leftwards at the horizontal groove 13, the crochet hook 6 at the top exposes from the top of the first suture needle 11.

FIG. 5 is the zoom-in picture of the crochet hook 6, the inside of the hook can only fit one seam.

The basis material of the seam is using the safe polyester fiber knitting thread 2-0, at least included a guiding zone and a functional zone, the guiding zone is located at one or two sides of the seam, processed by the adhesive to strengthen toughness, conquered the drawbacks of relatively soft of the traditional seam, and is convenient for operating under the inner mirror, the shop sign of the adhesive were non-poison adhesive of silicon 288; the functional zone is not processed by the adhesive, keeping the original pliability, is good for tying and fixing the parenchyma. The guiding zone and the functional zone could distinguish by colors.

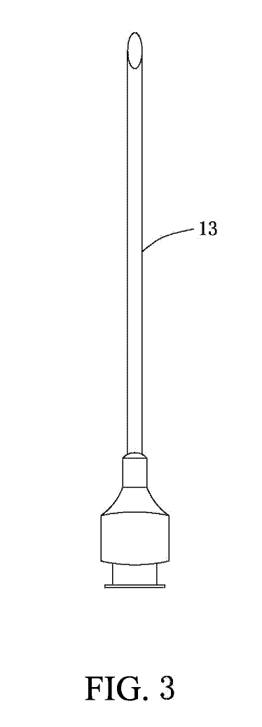

FIG. 3 is the third suture needle, and is also made from No. 12 puncture needle.

As one of the preferable embodiments of the present invention, a small hole 8 could be in the small handle 7 to strengthen the safety of the present invention, a line passing through the hole during the surgery, tied on the hand of the doctor, preventing the accidental slip during suture. A visible small hole could also be in the valve panel 3, in order to better inspect the entering situation of the inner core.

The present invention could also add some humanity details: the big handle 1 could be made in a certain roughness, preventing the slip during the surgery; in order to use integrated in more convenience, the big handle and the small handle could be made in the same color, being clear at a glance during the surgery.

During the operating, (1) Using the third suture needle (FIG. 3) to puncture in the upper joint compartment according to the certain puncturing point, meanwhile, the assistant holding the joint mirror, the operator puncturing in the joint disk at the junction point which is the outer one of the ⅓'s trisection from outside to inside at the junction line of the posterior band and the bilaminar region. Firstly going downward, and then going inside backward upper to puncture in the tissue of the juncture, and from the inside to puncture out at the ⅓ of the juncture point; (2) Prescribing the patient to keep their mouths opening, one hand of the assistant holding the joint mirror to keeping stable vision, the other hand of the assistant holding the third suture needle (FIG. 3) upward down to resist the back of condylar element, stabilize the joint disk; (3) Inserting the first suture needle (FIG. 1A) and the first inner core (FIG. 1B) to the patient's acoustic duct incision, pay attention to that the suture needle should not puncture the cartilage. Then puncturing in the seam, adjusting the position of the pinpoint, passing the seam through the wire snare 4, turning the first inner core (FIG. 1B) right or left 90 degrees locked, stringing up the seam. Meanwhile, backing off the pinpoint of the first suture needle (FIG. 1A), for the convenience of the seam's pulling out from the acoustic duct in the wire snare 4, pulling out the seam, then half needle suture done; (4) Putting the third suture needle (FIG. 3) backward to the puncturing point of the joint disk, and then putting it back upper into the recess, adjusting the length of the seam, the assistant putting the third suture needle (FIG. 3) forward down pressing the joint disk, for the convenience of the operator to puncturing in the second suture needle (FIG. 2A) at the patient's acoustic duct incision. The top crochet hook 6 of the second inner core (FIG. 2B) hooking the seam, leading it out of the joint compartment to the acoustic duct, finishing the first suture of the crewel traction suture fixation method of the joint disk; and (5) Inspecting the restoring situation of the joint disk under the inner mirror to find out whether need to suture continuously and the suturing position.

What is claimed is:

1. A suture fixation arthroscope apparatus of the temporomandibular joint (TMJ) disk, characterized in that: comprising a first suture needle and a first inner core, a second suture needle and a second inner core and a third suture needle, wherein:
the tail of the first suture needle is connected with a big handle via a valve panel, a vertical groove is opened towards a circle center of the surface of the big handle, a horizontal groove which is the same size as the vertical groove is vertically disposed in the place of the big handle connecting the valve panel, the first inner core comprises a seamless tube having an inner diameter less than the first suture needle, a wire snare protruding at both ends is welded at the top of the seamless tube, the radian angle is 90 degrees near the tail of seamless tube, a small handle is connected at the tail of the seamless tube, the first inner core can pass through the vertical groove of the big handle of the first suture needle, the corner of the 90 degrees near the tail of the seamless tube is able to lock rightwards or leftwards at the horizontal groove, and the wire snare at the top of the seamless tube exposes from the top of the first suture needle;
the tail of the second suture needle is connected with another big handle via a valve panel, a vertical groove is opened towards the circle center of the surface of the big handle, a horizontal groove which is the same size as the vertical groove is vertically disposed in at the place of the big handle connecting the valve panel, the second inner core comprises a seamless tube having inner diameter less than the second suture needle, a crochet hook the same size as the inner diameter is welded at the top of the seamless tube, the radian angle is 90 degrees near the tail of the seamless tube, a small handle is connected at the tail of the seamless tube, the second inner core can pass through the vertical groove of the big handle of the second suture needle, the corner of the 90 degrees near the tail of the seamless tube is able to lock rightwards or leftwards at the horizontal groove, and the crochet hook at the top of the seamless tube exposes from the top of the second suture needle; and
the third suture needle is a normal No. 12 puncture needle.

2. The suture fixation arthroscope apparatus of the temporomandibular joint (TMJ) disk according to claim 1, characterized in that: the inner diameter of the first protruded end of the two protruded ends wire snare is smaller than the body of the seamless tube, and the inner diameter of the second protruded end is greater than the body of the seamless tube.

3. The suture fixation arthroscope apparatus of the TMJ disk according to claim 1, characterized in that: the inside of the hook of the crochet hook can exactly fit one seam.

4. The suture fixation arthroscope apparatus of the TMJ disk according to claim 3, characterized in that: the basis material of the seam is made of polyester fiber knitting thread 2-0, at least comprises a guiding zone and a functional zone, the guiding zone located at one or two sides of the seam is processed by the adhesive, and the functional zone is not processed by the adhesive.

5. The suture fixation arthroscope apparatus of the TMJ disk according to claim 1, characterized in that: a small hole is in the middle of the small handle.

6. The suture fixation arthroscope apparatus of the TMJ disk according to claim 1, characterized in that: a visible small hole is in the middle of the valve panel.

7. The suture fixation arthroscope apparatus of the TMJ disk according to claim 1, characterized in that: the big handle and the small handle have a predetermined roughness.

8. The suture fixation arthroscope apparatus of the TMJ disk according to claim 1, characterized in that: the colors of the big handle of the first suture needle and the small handle of the first inner core are the same, and the colors of the big handle of the second suture needle and the small handle of the second inner core are the same.

9. The suture fixation arthroscope apparatus of the TMJ disk according to claim 1, characterized in that: the inner diameter of the first suture needle is slightly greater than the inner diameter of the second suture needle.
